(19) United States
(12) Patent Application Publication
(10) Pub. No.: US 2016/0038330 A1
(43) Pub. Date: Feb. 11, 2016

(54) SPINE PROTECTIVE AND CORRECTIVE EQUIPMENT

(71) Applicant: iCare121, inc, Palo Alto, CA (US)

(72) Inventor: Hyean Sung Kim, Daejeon (KR)

(73) Assignee: ICARE121, INC, Palo Alto, CA (US)

(21) Appl. No.: 14/633,144

(22) Filed: Feb. 27, 2015

(30) Foreign Application Priority Data
Aug. 11, 2014 (KR) 10-2014-0103800

(51) Int. Cl. A61F 5/02

(52) U.S. Cl. CPC A61F 5/026

(57) ABSTRACT

A spinal protective and corrective equipment is disclosed that is equipped with a zipper in front and a garment of resilient material worn on the part of body from the shoulders and to the hip; provided at the outside of the garment wrapping waist region with form of belt. The first elastic band is provided to compress the first lumbar spinous process. The second lumbar elastic band is provided to compress the second lumbar spinous process. The third lumbar elastic band is provided to compress the third lumbar spinous process. The fourth lumbar elastic band is provided to compress the fourth lumbar spinous process. The fifth lumbar elastic band is provided to compress the fifth lumbar spinous process.

The present invention relates to spine protective and corrective equipment, and more particularly, to spine protective and corrective equipment worn on body to hold a correct posture of the spinal vertebrae.

### BACKGROUND

The spine plays an extremely important role in our bodies; it supports the upper body's weight and provides posture while allowing for movement and flexibility. It also protects the spinal cord. The spine is the skeletal framework of the body, which supports our bodies and acts as a pillar. As shown in FIG. 1, it is composed of 5 parts: cervical (neck), thoracic (mid back), lumbar (lower back), sacral and coccygeal.

The normal spine has an “S” shaped curve when it is viewed from the side. The cervical and lumbar sections curve forward. The thoracic and sacrum sections curve backward. Kyphosis is an exaggerated rounding of back when the backward curvature of thoracic section is increased or the curvature of cervical and lumbar sections is abnormal.

If the forward curvature of lumbar is normal, the center of gravity is located rear side of the vertebrae. The supporting weight of the body is placed on the back of the spinal vertebrae and the articular surface, and fixed physiological effect is obtained.

When the lumbar spine is straight or curves outward, the central line of vertebrae moves forward and the center of gravity moves the upward nucleus pulpous, and the vertebrae is placed in an unstable position. The nucleus pulpous tends to move toward to the rear side of the vertebrae, where the boundary is relatively weak, when the forward weight presser disappears the posterior joints are abnormally open.

As a result, this may easily cause Facet joint syndrome, spinal hernia, and scoliosis. So, in case of Kyphosis or straightened spine it is important to correct the curvature of lumbar vertebrae. The pelvis is composed of Sacrum, Ilium, Pubis, Ischium and Coccyx. The most important bones are Sacrum and Ilium.

The sacroiliac joint is the joint in the bony pelvis between the sacrum and the ilium of the pelvis. When we stand, this joint moves weight of trunk and upper body to Ilium and Ischium. When we sit down it moves to lower body. It plays an important role as a plane joint which supports the spine.

Movement of the sacroiliac joint is not large as other joints. But it moves toward various directions like forward rotation, backward rotation, upward movement, downward movement or torsion. Thus, movement of the pelvis is fine, but because it has a variety of movement, pelvis structure can be deviated due to wrong posture. If it is deviated, the spinal vertebrae's upper structure become twisted and bent, resulting in scoliosis, shoulder height difference, and an unbalanced scapula.

In addition, the structure of the pelvis does also affect the lower body and it may cause length difference of legs, flat feet or toeing out. In order to prevent and cure these symptoms, it is important to tighten firmly sacroiliac joints. As described above, the lumbar spine and the sacroiliac joints are very important parts of our body.

However, the conventional spine protective devices worn on the upper body include waist straps, reinforcement plate for back support, waist and sacrum part and reinforcement garment for abdomen. But, they have a limitation to enhance vertebral joints since they do not support each of 5 lumbar vertebral bones and sacroiliac joint with sufficient strength.

### SUMMARY

In one aspect, a spine protective and corrective equipment provides a zipper in front and resilient material of the garment worn from shoulder to the hip which is provided at the outside of the garment surrounding waist and includes the first lumbar elastic band to compress the first lumbar spinous process, the second lumbar elastic band to compress the second lumbar spinous process, the third lumbar elastic band to compress the third lumbar spinous process, the fourth lumbar elastic band to compress the fourth lumbar spinous process and the fifth lumbar elastic band to compress the fifth lumbar spinous process.

In another aspect, the system provides a spine protective equipment where each individual belt compresses the spinous process of each of five lumbar vertebrae and supports spinal joints of adjacent lumbar vertebrae. The system compresses sacroiliac joint by using the belt wrapping sacral region and enhances the function of the sacroiliac joints. Further, the system provides a spine protective and corrective equipment to prevent and treat kyphosis by maintaining and increasing the curvature of lumbar.

Implementations of the above aspects can include one or more of the following. To compress sacroiliac joints it includes sacroiliac joint elastic band provided outside of the above garment, surrounding sacral region. In addition, it includes spine backboard support, which is provided at outside back of this garment; upper part of this curves backward and lower part curves forward and this will support the pillar of spine. The first lumbar elastic band, the second lumbar elastic band, the third lumber elastic band, the fourth lumber elastic band, the fifth elastic band and sacroiliac elastic band will be attached to this. In addition, the first lumbar elastic band spine, the second lumbar elastic band, the third lumbar elastic bands, the fourth lumbar elastic band, the fifth lumbar elastic band and sacroiliac joint elastic band are attached to one side of the spine support and the other surface of the spine support in contact with the garment will have five cushion members. Each of lumbar spinous process is compressed with dual pressure when each of the five cushion members corresponds to the respective mounting position of the lumbar spine and each of the lumber elastic band and each cushion member make a pair. Also, inside of the spine backboard support it includes the first forward curvature of spine backboard support with cushion form, which increases and maintains the first forward curvature of lumbar vertebrae. Further, it includes the second curvature of spine backboard support, which is attached to each of left and right side of region of the lower forward curvature. This increases and maintains the curvature of lumbar vertebrae supporting lumbar vertebrae and around lumbar vertebrae. Further, each of the five lumbar elastic bands can compress tightly each lumber spinous process fastening a buckle, which is consisted of the socket member of the end and to the plug member of the other side. Further, each of the five lumbar elastic bands includes a winding member, which winds a string connected to the one end and a string connected the other end. Also, each of them includes a rotating member attached to a winding member. Each lumbar spinous process can be compressed by wrapping waist tightly through rotating the winding member in the forward direction and winding strings of both ends at the winding member. In addition, each of the five lumber elastic bands has the first Velcro (hook and loop fastener) member to the one end and the second Velcro member to the other end. Each lumbar spinous process can be compressed by wrapping waist firmly through attaching the first Velcro member to male Velcro or female Velcro, which is attached to the other side with the sewing conditions and attaching the second Velcro member to male Velcro or female Velcro which is attached to the other side with the sewing conditions. Further, the first Velcro member is connected to the one side of this sacroiliac elastic band and the second Velcro member is connected to the other side of the sacroiliac elastic band. The sacroiliac joint can be compressed by pulling the first Velcro member, attaching it to the female Velcro or male Velcro with sewed conditions and pulling the second Velcro member, attaching it to the female Velcro or male Velcro with sewed conditions.

Advantages of the invention may include one or more of the following. The system improves the joint function of adjacent lumbar vertebrae by compressing each lumbar spinous process using each belt, which wraps waist region and each lumbar vertebrae. And, it has an effect of preventing lumbar kyphosis by maintaining and increasing the forward curvature of lumbar vertebrae. The protective equipment supports the spinal joints while maintaining and increasing spinal curvature and compressing sacral iliac joint with enough pressure. The spine protective and corrective equipment has effect of supporting vertebral joints adjacent lumbar vertebrae compressing each spinous process using each individual belt, which surrounds waist region and each lumbar vertebra. Further, it can enhance the sacroiliac joints by compressing sacroiliac joints with belt surrounding sacral region. In addition, the system prevents lumbar kyphosis increasing and maintaining the forward curvature of lumbar vertebrae.

### BRIEF DESCRIPTION OF THE DRAWINGS

FIG. 1 is a side view of a normal spine.

FIGS. 2a and 2b are perspective views of spine protective and corrective equipment in accordance with an embodiment of the present invention.

FIG. 3a is a perspective view of the present invention: the second front curvature backboard support on both sides of the spine backboard support.

FIG. 3b is a perspective view of spine backboard support of this invention.

FIG. 3c is a side view of spine backboard support of this invention.

FIG. 4 is a perspective view of the spine protective and corrective equipment according to another embodiment of this invention.

FIG. 5 is a perspective view of the spine protective and corrective equipment according to another embodiment of this invention.

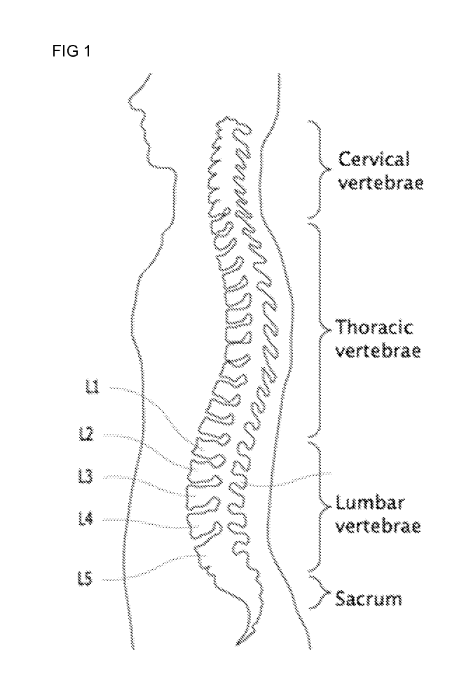

### DESCRIPTION

Hereinafter, with reference to the accompanying drawings, an embodiment according to the present invention will be described in more detail.

For purposes of description herein, the terms “upper,” “lower,” “right,” “left,” “rear,” “front,” “vertical,” “horizontal,” and derivatives thereof shall relate to the system as described in the figures. However, it is to be understood that the invention may assume various alternative orientations, except where expressly specified to the contrary. It is also to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as unnecessarily limiting, unless the claims expressly state otherwise.

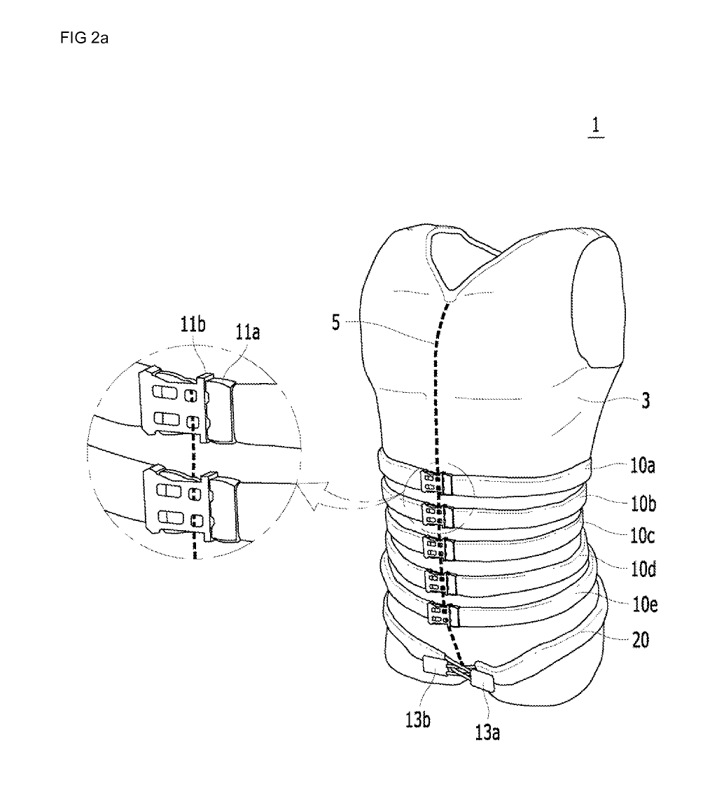

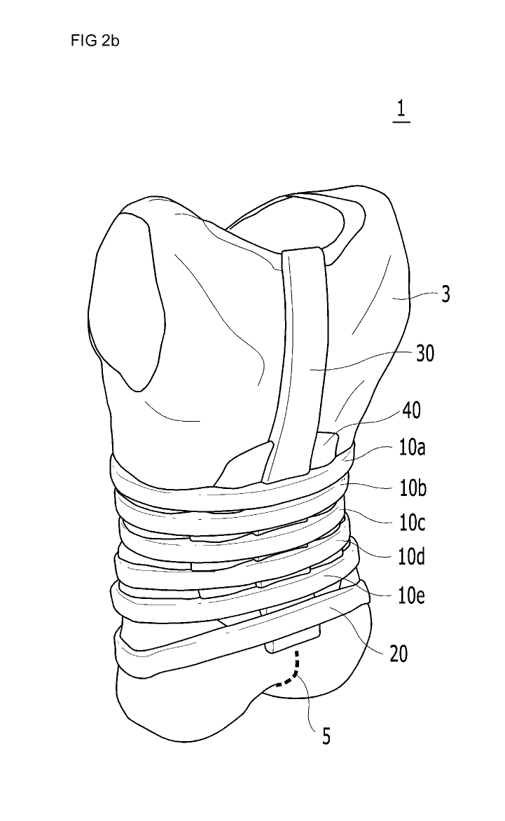

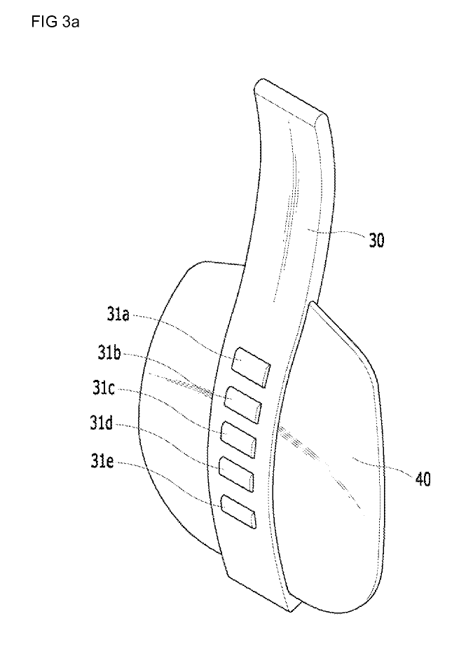

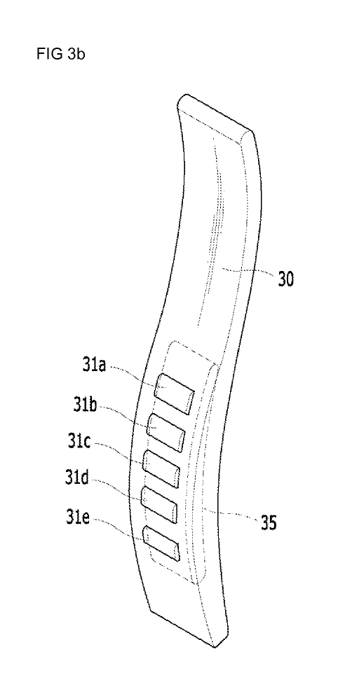

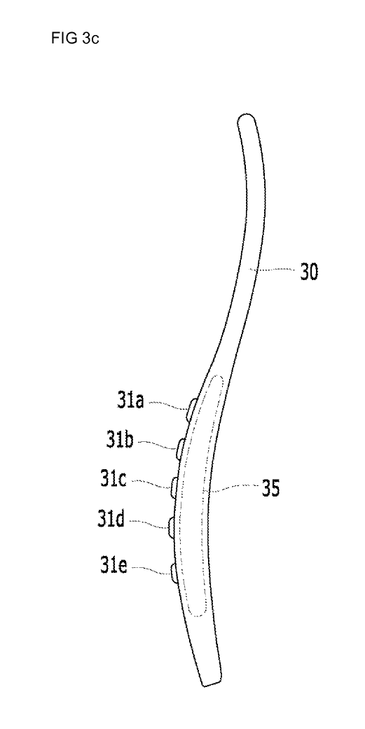

Referring to FIGS. 2a to 3c, spine protection and correction tools (1) include the garment of resilient material (3), the first lumbar elastic band (10a), the second lumbar elastic band (10b), the third lumbar elastic band (10c), the fourth lumbar elastic band (10d) and the fifth lumbar elastic band (10e), a sacroiliac joint elastic band (20) and a spine support (30), the five cushion member (31a-31e), the first forward curvature of spine backboard support (35) and the second forward curvature of spine backboard support (40).

The garment of resilient material (3) has a zipper (5) in front and is worn from the shoulders to the hip. This supports a healthy spinal function by wrapping spine and related structures of spine and sacroiliac joints by giving support them. The material of the garment is resilient and sweat-proof with excellent physical contact. The front zipper of clothing (5) will be a detachable structure, which is composed of various structures of one, two and three frame structure.

The first lumbar elastic band (10a) is provided at the outside of the garment (3). It is a form of belt encompassing the waist. It compresses the first lumbar (L1) spinous process and supports the function of the joint between the first and the second lumbar vertebrae consisting of elastic material band.

The second lumbar elastic band (10b) is provided under the first lumbar elastic band (10a) of the garment (3). It is a form of belt encompassing the waist. It compresses the second lumbar (L2) spinous process and supports the function of the vertebral joint between the second and the third lumbar vertebrae. It is made of resilient material band.

The third lumber elastic band (10c) is provided under the second lumbar elastic band (10b) of the garment (3). It is a form of belt encompassing the waist. It compresses the third lumbar (L3) spinous process and supports the function of the joint between the third and the fourth lumbar vertebrae. It is made of resilient material band.

The fourth lumber elastic band (10d) is provided under the third lumbar elastic band (10c) of the garment (3). It is a form of belt encompassing the waist. It compresses the fourth lumbar (L4) spinous process and supports the function of the joint between the fourth and the fifth lumbar. It is made of resilient material band.

The fifth lumber elastic band (10e) is provided under the fourth lumbar elastic band (10d) of the garment (3). It is a form of belt encompassing the waist. It compresses the fifth lumbar (L5) spinous process and supports the function of the joint between the fifth lumbar vertebrae and the first sacral vertebrae. It is made of resilient material band.

Each of five lumbar elastic bands (10a-10e) presses each of five lumbar spinous processes (L1-L5) by tightening a fastening buckle, which is composed of the socket member (11b) coupled to the one end and the plug member (11a) coupled to the other end. Weakened joints can be reinforced and maintained by tightening the fastening buckles. The fastening buckles are made of plastic material.

Sacroiliac joint elastic band (20) is provided at the outside of the garment (3) with a form of belt encompassing sacral region and compresses the sacroiliac joints. It prevents several symptoms caused in sacroiliac joints by tightening sacroiliac joints. This is a resilient material band.

Sacroiliac joint elastic band (20) is connected to the first Velcro member (13a) on the one end and connected to the second Velcro member (13b) on the other end. It reinforces sacroiliac joints by pulling the first Velcro member (13a) and attaching to the male or the female Velcro, and by pulling the second Velcro member (13b) and attaching to female Velcro or male Velcro attached. The first Velcro member (13a) and the second Velcro member (13b) cross each other and can be adjusted to suit body size by pulling in opposite directions.

The spine backboard support (30) is provided in the middle of outside of the back. Upper part is curved backward and lower part is curved forward. This supports the column of the spinal vertebrae. The first lumbar elastic support band (10a), a second resilient band spine (10b), the third lumbar elastic band (10c), the fourth lumbar elastic band (10d), the fifth lumbar elastic band (10e) and the sacroiliac joint elastic band (20) are attached to the part curved forward in order from above. Spine backboard support (30) preferably is made of a strong material for supporting the spinal column to allow the sufficient strength to recover its original shape. The lower part is curved forward and has a function to maintain the curvature of lumbar vertebrae.

The first lumbar elastic band (10a), the second lumbar band spine (10b), the third lumbar elastic band (10c), the fourth lumbar elastic band (10d), the fifth lumbar elastic band (10e) and the sacroiliac joint elastic band (20) are attached to the one side (outside) of the spine backboard support (30). Five cushion members (31a-31e) of convex shape pressing each spinous process are attached to the other side (inside) of the spine supporting part (30) abuts the garment (3)

Each of the five cushion members (31a-31e) is attached to the one surface of each of the elastic band of five lumbar vertebrae (10a-10e) so as to correspond to the respective mounting position of the lumbar spine. Pairs of each elastic band (10a-10e), and each cushion member (31a-31e) press the lumbar spinous processes with dual pressure. For example, when the third lumbar elastic band (10c) at the back forces to the lumbar spine with pressure, this force will be delivered to the third cushion member (31c) and convex shape of the third cushion member (31c) will press the third lumbar spinous process (L3).

Thus, it is very important to give each individual pressure to each lumbar spinous process. Since bones of our bodies are getting better by electric action with stimulation, when each of the cushion members presses the corresponding each individual lumbar spinous process, the spinal vertebrae will be strong and the spinal joint function of the lumbar vertebrae can be enhanced.

The first forward curvature of spine backboard support of (35) is provided inside of the forward curvature along the longitudinal direction of the spine backboard support (30). It is preferably made of the cushion material, which has enough strength to increase and maintain the forward curvature.

The second forward curvature (40) of spine backboard support is a form of wing combining the left and right part at lower part of the forward curvature of the spine backboard support (30). This increases and maintains the forward curvature of the lumbar vertebrae by supporting the lumbar vertebrae and surrounding area of lumbar vertebrae. It is preferably made of strong material enough to increase and maintain the forward curvature.

The first resilient band spine (10a), the second resilient band spine (10b), the third lumbar elastic band (10c), the fourth lumbar elastic band (10d) and the fifth lumbar elastic band (10e) are attached to the one side (outer space) of the spine backboard support (30) and force to the spine direction. Thus, the first forward curvature of the backboard support (35) and the second forward curvature of the backboard support (40) are enforced by the lumbar elastic bands (10a-10e). Thus, the forward curvature of lumbar is increased and maintained.

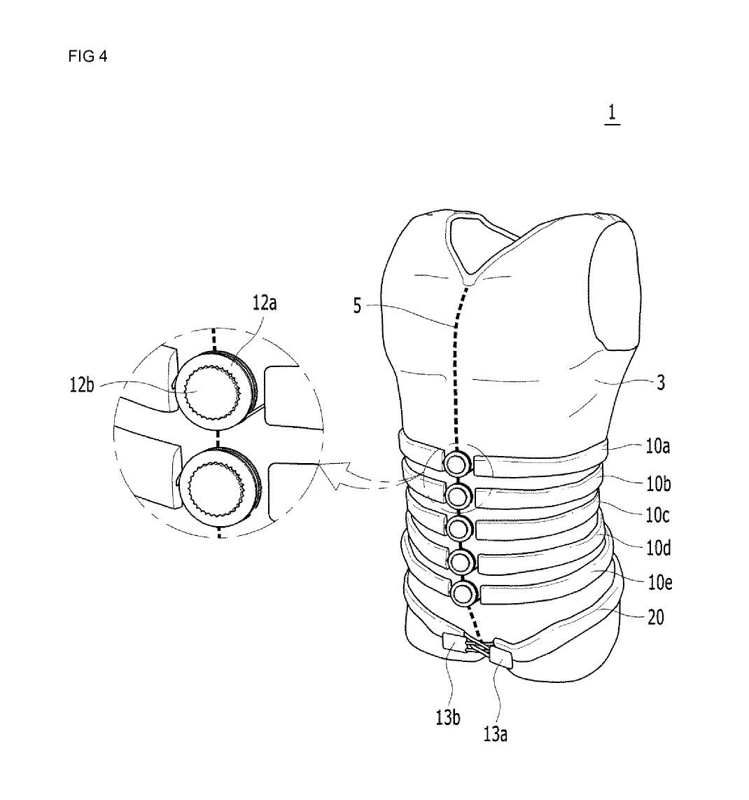

Referring to FIG. 4, each of the five lumbar vertebrae elastic bands (10a-10e) includes the rotating member (12b) attached to the winding member (12a). Each lumbar spinous process can be compressed by rotating the rotating member (12b) in the forwarded direction and both ends of the string are winded. When the rotating member (12b) is rotated in the reverse direction, strings of both ends are released from the winding member (12a).

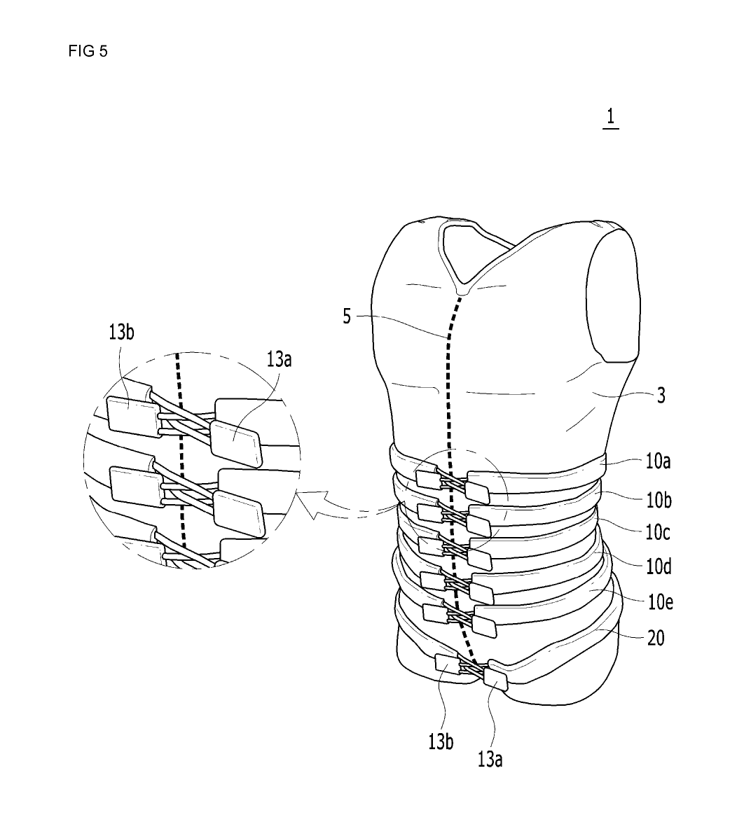

Referring to FIG. 5, each of the five lumbar vertebrae elastic bands (10a-10e) is connected to the first Velcro member (13a) at the one end and the second Velcro member (13b) at the other end. Each spinous process is compressed by wrapping waist region firmly through attaching the first Velcro member (13a) to the female Velcro or the male Velcro attached at the one end with being sealed.

According to the present invention, there is an effect of augmenting the function of the vertebral joint adjacent lumbar vertebrae by compressing the each spinous process by using each individual belt, which wraps waist region and each lumber vertebra. Further, there is an effect of augmenting the sacroiliac joints by compressing the sacroiliac joints by using the belts, which wraps the sacral area.

In addition, there is an effect of preventing the lumbar kyphosis by maintaining and increasing the curvature of the lumbar vertebra.

The present invention is not limited in the above specific preferred embodiments. Those skilled in the art will readily appreciate that various modification whether specifically or expressly identified herein without departing from the above teachings or scope of this technical disclosure. Accordingly, all such modifications are contemplated and intended to be included with the scope of the claims.

A summary of element numbers used in the drawings is provided below:

| | |
|---|---|
| 1: Spine protective and | 3: Garment of resilient material |
| corrective equipment | |
| 5: zipper | 10a-10e: Lumbar Elastic Bands |
| 11a: the plug member | 11b: the socket member |
| 12a: the winding member | 12b: the rotating member |
| 13a: The first Velcro member | 13b: The second Velcro member |
| 20: sacroiliac joint elastic band | 30: Spine backboard support |
| 31a-31e: cushion member | 35: The first retaining portion lordosis |
| 40: The second retaining portion | |
| lordosis | |

It is to be understood that the foregoing description is intended to illustrate and not to limit the scope of the invention, which is defined by the scope of the appended claims. Other embodiments are within the scope of the following claims. For example, while the ballistic lumbar support belt has been described as including various ballistic resistant materials, it may include any other type of ballistic-resistant material or combination thereof. Further, while the ballistic resistant material was described as being of a particular denier, areal density and thickness, ballistic resistant material having any denier, areal density or thickness may be used.

What is claimed is:

1. A spine protective and corrective equipment, comprising:
a zipper provided on a front of a body and a resilient material garment worn from shoulders and to the hips;
a first lumbar elastic band provided at the outside of the garment with a belt surrounding a waist to compresses the first lumbar spinous process;
a second lumbar elastic band compresses the second spinous process;
a third lumbar elastic band compresses the third spinous process;
a fourth lumbar elastic band compresses the fourth spinous process; and
a fifth lumber elastic band compresses the fifth spinous process

2. The method of claim 1, comprising a sacral joint elastic band provided at an outer side of the garment wrapping sacral region with a belt.

3. The method of claim 2, comprising a spine backboard support at an outside of the garment and in the center of the back, the spine backboard support having an upper part being backward curved and a lower part being forward curved and supporting the spinal column, wherein the first lumbar elastic band, the second lumbar elastic band, the first lumbar elastic band, the fourth lumbar elastic band, the fifth lumbar elastic band and the sacral elastic band are attached to the spine backboard support.

4. The method of claim 3, wherein the first lumbar elastic band, the second lumbar elastic band, the third lumbar elastic band, the fourth lumbar elastic band, the fifth lumbar elastic band and the sacroiliac joint elastic band are attached to the one side of the spine backboard support and five cushion members compressing each spinous process are attached to the other side of the spine backboard support, wherein each pair of each cushion members and each lumbar elastic band attached to the spine backboard support to provide dual pressure to each lumber spinous process.

5. The method of claim 3, wherein an inside of a forward curved shape of the spine backboard support, a spine supportive and corrective equipment includes further the forward curvature of spine backboard support which increases and supports the forward curvature with cushion.

6. The method of claim 3, comprising a spine supportive and corrective tool includes further the second forward curvature of spine backboard support, which increases and supports the forward curvature of the lumber by being attached to the left and right in the lower part of the forward curvature in the spine backboard support and by supporting the waist region of the lumbar and around the lumber.

7. The method of claim 1, wherein each of the five lumbar elastic bands compresses each lumbar spinous process by wrapping the waist region through tightening each fastening buckle which is consisted of the socket member attached to one end and the plug member attached to the other end.

8. The method of claim 1, wherein each of the five lumbar elastic bands includes a string attached to one end, the winding member attached to the other end and a rotating member. Each of the spinous process is compressed by rotating the rotating member in the forwarded direction so that string of both ends may be winded at the winding member and surrounds tightly the waist region.

9. The method of claim 1, wherein each of the five lumbar elastic bands is attached to a first hook and loop fastener (Velcro) member and a second Velcro member to the other end by attaching the first Velcro member to a female Velcro or male Velcros attached to the other end with being sealed and attaching the second Velcro member to a female and a male Velcro, and wrapping tightly a waist region, wherein each lumbar spinous process is compressed.

10. The method of claim 2, comprising a sacroliliac elastic band is attached to the first Velcro member on one end and the second Velcro on the other end, wherein a Sacroliliac joint is compressed by wrapping tightly a waist region through attaching the first Velcro member to the female Velcro or male Velcro attached to the other end with being sealed and attaching the second Velcro member to the female and male Velcro.

11. The method of claim 1, wherein the equipment is customized for individual user according to size, position of each cushion.

12. The method of claim 1, comprising a dial to set a power of pulling individual band as number in rotation member, belt and Velcro.
